(19) United States
(12) Patent Application Publication
(10) Pub. No.: US 2009/0099496 A1
(43) Pub. Date: Apr. 16, 2009

(54) PRESSURE BANDAGE WITH MEDICATION DELIVERY SYSTEM

(75) Inventors: Roger W. Heegaard, Minneapolis, MN (US); John K. Lampe, Saint Paul, MN (US); William G. Heegaard, Minneapolis, MN (US); Eric G. Heegaard, Saint Paul, MN (US)

Correspondence Address: SHERRILL LAW OFFICES, 4756 BANNING AVE, SUITE 212, WHITE BEAR LAKE, MN, 55110-3205, US

(73) Assignee: MEDTREO, LLC, Minneapolis, MN (US)

(21) Appl. No.: 12/089,133

(22) PCT Filed: Oct. 5, 2006
(86) PCT No.: PCT/US06/39353

§ 371 Date: Sep. 23, 2008

Related U.S. Application Data
(60) Provisional application No. 60/723,599, filed on Oct. 5, 2005.

(51) Int. Cl. A61F 13/00

(52) U.S. Cl. 602/53

(57) ABSTRACT

A pressure bandage (100) for applying pressure to a target site comprising (i) a backing layer (102) with a coating of a pressure-sensitive adhesive on a first major surface of the backing layer, and (ii) a snap-bucklable member (104) coupled to the backing layer an capable of snap-buckling from a concave to a convex configuration after the bandage has been adhesively applied over the target site so as to apply continuous pressure to the target site so long as the bandage is worn. The pressure bandage may be positioned over a bleeding target site to apply pressure to the target site and reduce bleeding.

This application claims the benefit of U.S. Provisional Application No. 60/723,599, filed Oct. 5, 2005.

### FIELD OF INVENTION

The present invention generally relates to medical bandages. Specifically, the invention relates to pressure bandages.

### BACKGROUND OF THE INVENTION

Bandages serve many functions. These functions can include protection of a wound from germs and contaminants, protection of a would from further trauma that could cause additional pain, immobilization or restriction of motion to promote healing, compression to reduce swelling or bleeding, creation of a better wound environment, such as maintaining a high level of moisture or applying medication to aid in healing, or concealment of a wound for cosmetic purposes.

The prior art reveals various kinds of pressure bandages. Pressure bandages are often used to administer pressure to a wound site. For example, U.S. Pat. No. 5,628,723 to Grau teaches a device with a pressure enhancement member for applying pressure to wounds to stop the flow of blood. Another patent, U.S. Pat. No. 5,823,983 to Rosofsky et al. describes a pressure bandage with a compressed cellulose layer that expands when wet.

The prior art suffers from certain shortcomings or limitations. The purpose of the present invention is to overcome these and other shortcomings or limitations in the prior art.

### SUMMARY OF THE INVENTION

A first aspect of the invention is a pressure bandage for applying pressure to a target site. The pressure bandage includes (i) a backing layer having a coating of a pressure-sensitive adhesive on a first major surface of the backing layer, and (ii) a snap-bucklable member coupled to the backing layer and capable of snap-buckling from a concave to a convex configuration after the bandage has been adhesively applied over the target site, whereby the convex snap-bucklable member applies continuous pressure to the target site so long as the bandage is worn.

A second aspect of the invention is a method of applying pressure to a bleeding target site, comprising the steps of (i) obtaining a pressure bandage, comprising (A) a backing layer having a coating of a pressure-sensitive adhesive on a first major surface of the backing layer, and (B) a snap-bucklable member coupled to the backing layer and capable of snap-buckling from a concave to a convex configuration, (ii) adhering the pressure bandage over a bleeding target site with the snap-bucklable member positioned over the target site, and (iii) snap-buckling the snap-bucklable member from a concave to a convex configuration.

### BRIEF DESCRIPTION OF THE DRAWINGS

FIG. 1 is a top view of one embodiment of the invention.

FIG. 2 is an exploded side view of the invention shown in FIG. 1.

FIG. 3A is a cross-sectional side view of the invention shown in FIG. 1, taken along line 3-3 with the dome-shaped member in a concave configuration.

FIG. 3B is a cross-sectional side view of the invention shown in FIG. 3A after the dome-shaped member has been inverted into a convex configuration.

FIG. 4 is a perspective view of a sheet from which a plurality of dome shaped members have been formed.

FIG. 5 is an exploded side view of a second embodiment of the invention.

FIG. 6 is an enlarged perspective view of a portion of the invention shown in FIG. 5 after assembly.

FIG. 7 is a cross-sectional side view of a third embodiment of the invention.

FIG. 8A is a side view of the dome-shaped member and seal portion of the invention shown in FIG. 7 with the dome-shaped member in a concave configuration so as to define a container.

FIG. 8B is a side view of the of the dome-shaped member and seal portion of the invention shown in FIG. 8A after the dome-shaped member has been inverted into a convex configuration and the seal broken so as to release fluid from the container.

FIG. 9A is a cross-sectional side view of a fourth embodiment of the invention with the dome-shaped member in a concave configuration.

FIG. 9B is a cross-sectional side view of the invention shown in FIG. 9A after the dome-shaped member has been inverted into a convex configuration.

### DETAILED DESCRIPTION OF THE INVENTION INCLUDING A BEST MODE

### Nomenclature

100 Pressure Bandage
101 Pad
102 Backing
103 Periphery of Bandage
104 Snap-Bucklable Member
106 Interior Side of Bandage
107 Exterior Side of Bandage
108 Sheet of Snap-Bucklable Material
109 Die Cut Line
200 Pressure Bandage
201 Pad
202 Backing
204 Snap-Bucklable Member
206 Interior Side of Bandage
207 Exterior Side of Bandage
210 Frangible Container of a Flowable Material
300 Pressure Bandage
302 Backing
304 Snap-Bucklable Member
306 Interior Side of Bandage
307 Exterior Side of Bandage
310 Container Defined by Snap-Bucklable Member
315 Seal
320 Flowable Material
400 Pressure Bandage
402 Backing
404 Snap-Bucklable Member
406 Interior Side of Bandage
407 Exterior Side of Bandage
425 Plunger
426 Covering
427 Plunger Face
X Longitudinal Direction
X_{Center} Longitudinal Centerline
Y Lateral Direction
Y_{center} Lateral Centerline
Z Transverse Direction
Z_{Center} Transverse Centerline
Z₁ First Transverse Direction

### DEFINITIONS

When a range or interval is disclosed, the disclosure is intended to disclose both the endpoints and the intervals within the range. For example, a range of 0.005 to 0.010 includes 0.005, 0.006 and 0.010 within that range.

### Construction

The invention is a medical device for bandaging parts of the human body. Specifically, the device is a bandage 100, 200, 300, 400 with a pressure mechanism.

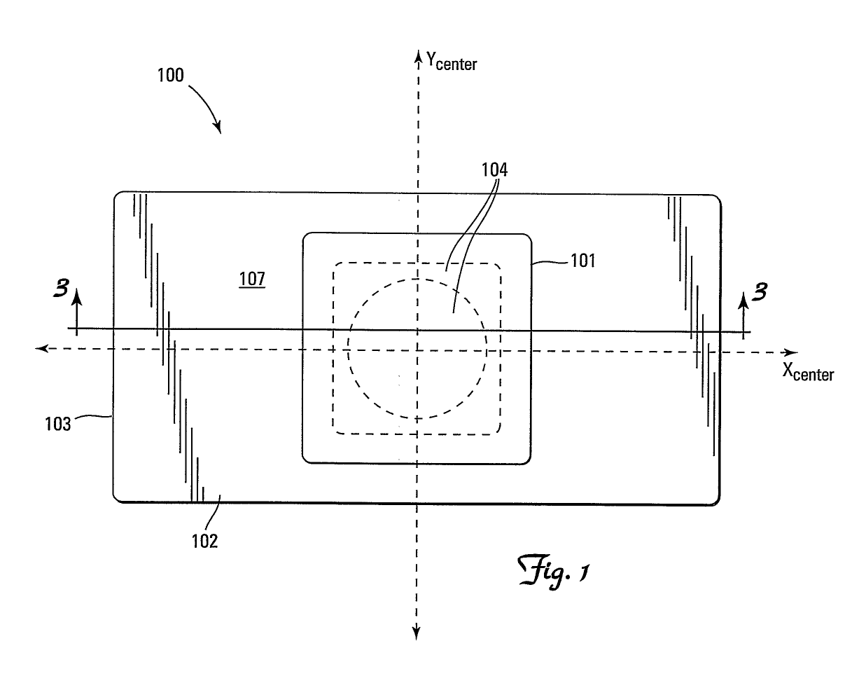

In one embodiment depicted in FIGS. 1, 2, 3A and 3B, the pressure mechanism is a dome-shaped snap-bucklable member 104 layered between the backing 102 and an absorbent pad 101 of an adhesive bandage 100. When the bandage 100 is applied to the target site (not shown) and when pressure is applied in the first transverse direction Z₁ to the snap-bucklable member 104 through the backing 102, the snap-bucklable member 104 snap-buckles into an inverted or convex position and remain in that inverted position. The inverted snap-bucklable member 104 applies pressure to the target site (not shown) covered by the bandage 100. The pressure created by the snap-bucklable member 104 can also be used to release beneficial substances such as medication.

### FIRST EMBODIMENT

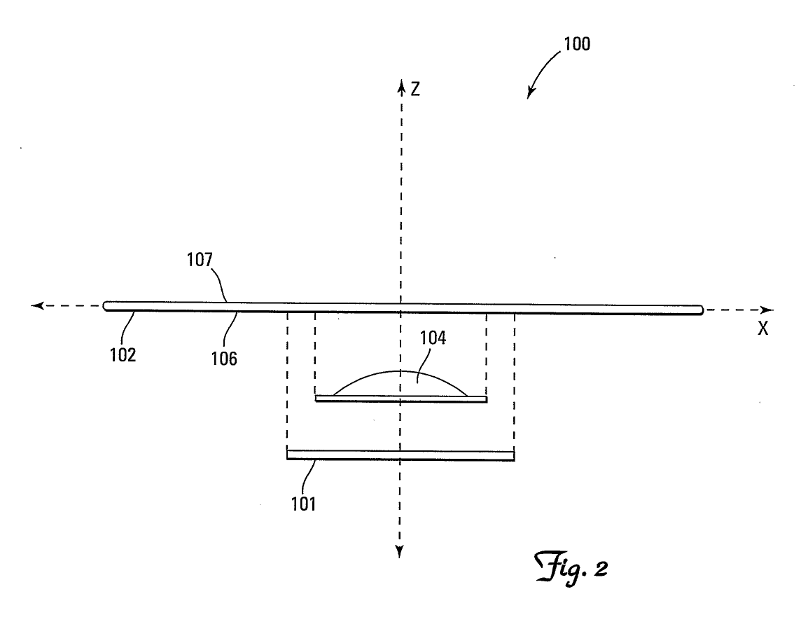

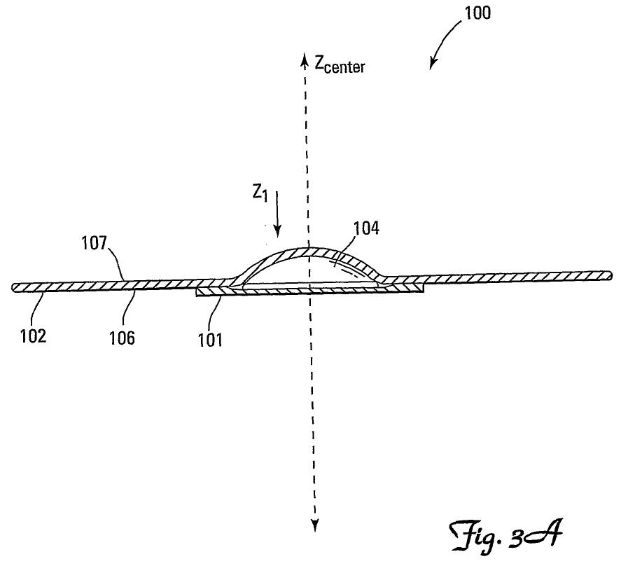

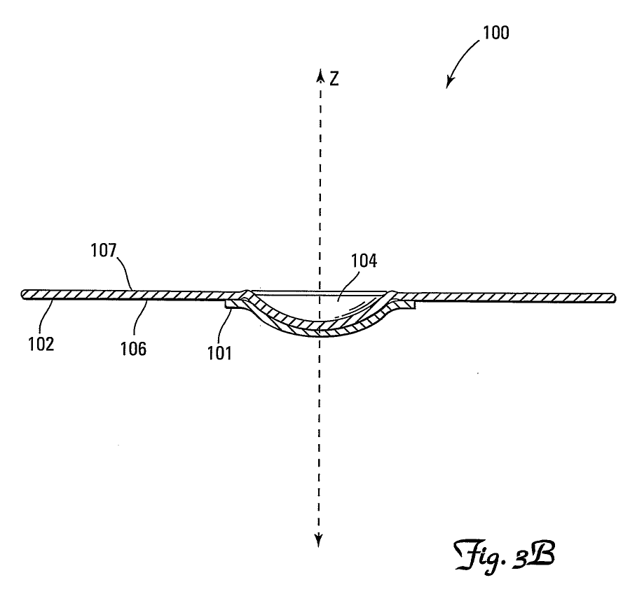

A first embodiment of the pressure bandage 100 is shown in FIGS. 1, 2, 3A and 3B. The bandage 100 can be oriented with a longitudinal centerline X_{Center} generally bisecting the bandage 100. The term “longitudinal” refers to a line, axis, or direction in the plane of the bandage 100 that is aligned with the longitudinal centerline X_{Center}. The bandage shown in FIG. 1 can further be oriented with a lateral centerline Y_{Center} that is perpendicular to the longitudinal centerline X_{Center}. The term “lateral” refers to a line, axis, or direction in the plane of the bandage 100 that is aligned with the lateral centerline Y_{Center}. The bandage 100 can further be oriented by a transverse centerline Z_{Center} as shown in FIGS. 2, 3A and 3B, which is perpendicular to the plane (unnumbered) formed by the longitudinal centerline X_{Center} and the lateral centerline Y_{Center}, and generally corresponds to the direction associated with the thickness dimension of the bandage 100. The shape of the bandage 100 is defined by its peripheral edge 103.

The length of the bandage 100 is the maximum dimension measured in the longitudinal direction X. The width of the bandage 100 is the maximum dimension measured in the lateral direction Y. The thickness of the bandage is the maximum dimension measured in the transverse direction Z.

The pressure bandage 100 as shown in FIGS. 1, 2, 3A and 3B has an interior side 106 that generally faces toward the body (not shown) of a wearer (not shown) of the bandage 100. The bandage 100 also has an exterior side 107 that generally faces away from the body (not shown) of the wearer (not shown) of the bandage 100.

The pressure bandage 100 includes (i) a backing 102 coated on the interior side 106 with an adhesive of a kind well known in the art of bandages, and (ii) a snap-bucklable member 104, such as the dome shaped member depicted in the FIGs. The pressure bandage 100 may also include a pad 101 of a kind well known in the art of bandages.

The pressure bandage 100 can be applied to a target surface (not shown) such as the skin (not shown) of a human body (not shown) and held in place with the adhesive on the interior side 106 of the backing 102. Pressure in the first transverse direction Z₁ can then be applied by pressing with a finger (not shown) on the snap-bucklable member 104 as shown in FIGS. 3A and 3B. This pressure can cause the snap-bucklable member 104 to invert from a concave shape to a convex shape (i.e., snap buckle) as shown in FIG. 3B. By inverting the snap-bucklable member 104, pressure can be applied in the first transverse direction Z₁ to a target site (not shown), which is preferably bisected by the transverse centerline Z_{Center} of the bandage 100. The snap-bucklable member is preferably constructed, configured and arranged to remain inverted until the bandage 100 is removed.

The pad 101 can have different properties and be made in different ways. For most applications a film (not shown) can be provided on the interior side 106 of the pad 101 to provide various beneficial properties such non-adherent, non-absorbent, air and liquid permeable. Such qualities can prevent the pad 101 from sticking to a wound, can allow wound exudate to pass through to absorbent materials in the pad 101 and can regulate the level of moisture present at or near a wound site. One suitable material for such a film (not shown) on the pad 101, can be Delnet® Apertured Film from Delstar Technologies, Inc., of Middleton, Del. Materials such as Mylar® or other film materials or coatings can also be used to form a nonadherent layer (not shown).

The pad 101 can also have absorbent characteristics allowing it to absorb moisture and wound exudate. The absorbent properties are usually created with a second layer (not shown) in the pad 101 of a different material. For many applications nonwoven blends of rayon and polyester can be used. Other materials can be used for any absorbent material layer in the pad 101 including natural fibers such as cotton or woven materials or absorbent foams. Such modifications and other modifications that, for example, remove the pad 101 entirely or change the properties of the pad 101 such as making it entirely of nonabsorbent materials, are within the scope of this invention.

The backing 102 can be a stretchable material of a kind typically used with adhesive bandages. The backing 102 could be stretchable and breathable with an adhesive on the interior side 106 of the bandage 100. A suitable material can be a tape with a polyurethane backing and an acrylate adhesive such as elastic nonwoven tape, Part No. 9907, from 3M®, Inc., of St. Paul, Minn. Other suitable materials include specifically, but not exclusively, polyurethane films, polyolefin films, polyvinylchloride films, ethylene vinyl acetate films, woven fabrics, and nonwoven fabrics. Such modifications and other modifications that, for example, remove the backing 102 entirely or change the properties of the backing 102 such as making it entirely of non-stretchable materials, are within the scope of this invention.

The snap-bucklable member 104 can be made from any material capable of providing the necessary and desired snap-buckling effect. Suitable materials include specifically, but not exclusively thin plasticized plastics such as a clear polyester film available from Tekra Corporation of New Berlin, Wis. under the mark MELINEX 1311. Other suitable materials include specifically, but not exclusively, metals, cellulose based plastics and cardstock. Such modifications and other modifications that, for example, alter the shape of the snap-bucklable member 104 (some of which are described in other embodiments below), are within the scope of this invention.

The overall size and shape of the bandage 100 can vary depending on the application. A bandage 100 that is 2 inches long and 1 inch wide could be suitable for some applications such as stopping the bleeding of venous puncture wounds on the arm (not shown) from injections or catheters (not shown). For other applications, such as on the thigh (not shown), a bandage 100 that is 8 inches long by 4 inches wide could be suitable. Other lengths and widths could also be suitable. The appropriate size could largely depend on the part of the body for which the bandage 100 is intended and the amount of pressure needed.

The shape of the bandage 100 can also vary depending on the application. While the bandage 100 shown in FIG. 1 is rectangular in shape, the bandage 100 may take on any of the various shapes well known in the art of bandages.

The bandage 100 can be held onto a surface (not shown) such as the skin (not shown) in different ways. The bandage 100 disclosed here uses adhesive on the interior side 106 of the backing 102. However, various other means of fastening including hook and loop fasteners, straps, buttons, hooks, etc., in combination with wraps, for example, can be employed. Such modifications can still be within the scope of this invention.

The manufacture of the bandage 100 can be accomplished in a variety of ways. For many applications, it can be preferable to have some portion or all of the cutting and assembly of the bandage 100 completed using a web converting process. The pad 101 and the backing 102 can be cut using a rotary die and bonded to each other using adhesives in such a process.

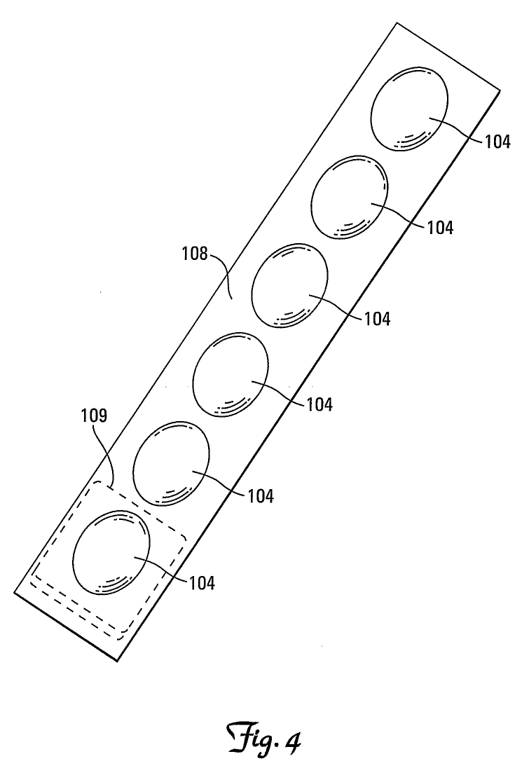

The snap-bucklable member 104 can also be manufactured and placed into the bandage 100 using a web converting process. FIG. 4 shows a sheet 108 of material suitable for use in forming snap-bucklable members 104, such as PET. The snap-bucklable members 104 can be conveniently formed in a stamping operation (with the application of heat). The snap-bucklable member 104 can then be cut along line 109 and inserted between the backing 102 and the pad 101 during the web converting process.

The bandage 100 can be constructed and manufactured in many different ways. Although a web converting process is described above, various manufacturing techniques including assembly by hand can be employed. Bonding techniques other than adhesives can be used. For example, heat can be used to melt layers together or mechanical fasteners can be used to fasten components together. Such modifications are still within the scope of this invention.

The bandage 100 as disclosed above can have many uses and offer several advantages including the following. First, the bandage 100 can be a more sterile and more efficient means to apply light pressure to wound sites to stop bleeding. For example, when a patient receives an injection such as for immunization, a venous puncture wound is typically created. Oftentimes, a caregiver will combine a cotton ball and tape to apply pressure to the wound site (not shown). A pressure bandage 100 (that can be efficiently sterilized during manufacture) can be used instead of tape and a cotton ball for wound sites such as venous puncture wounds.

Second, the bandage 100 can be used for various other reasons, such as limiting blood flow. For example, to limit blood flow to the site of an incision, the pressure bandage 100 can be applied to a vein or artery.

Other embodiments can encompass bandages 100 with different characteristics and properties and can incorporate many of the characteristics and properties described above. Some of those other embodiments are discussed below.

### Second Embodiment

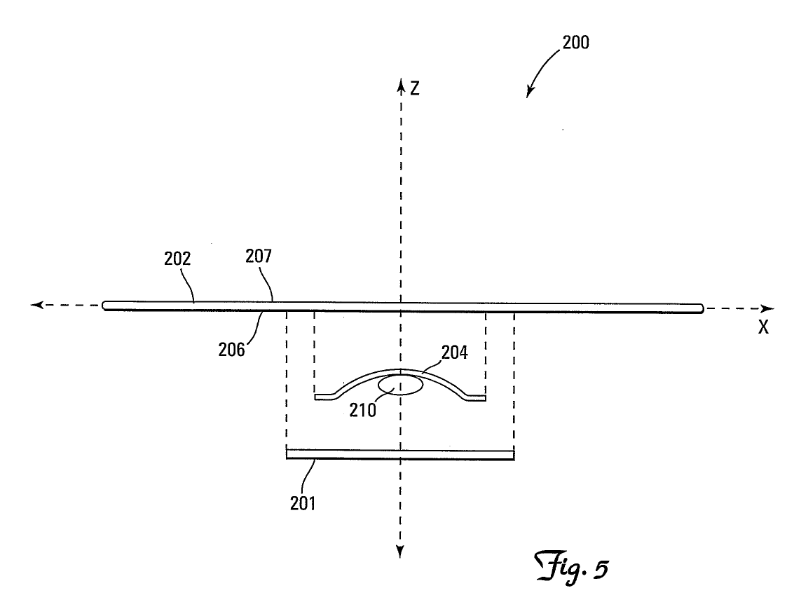

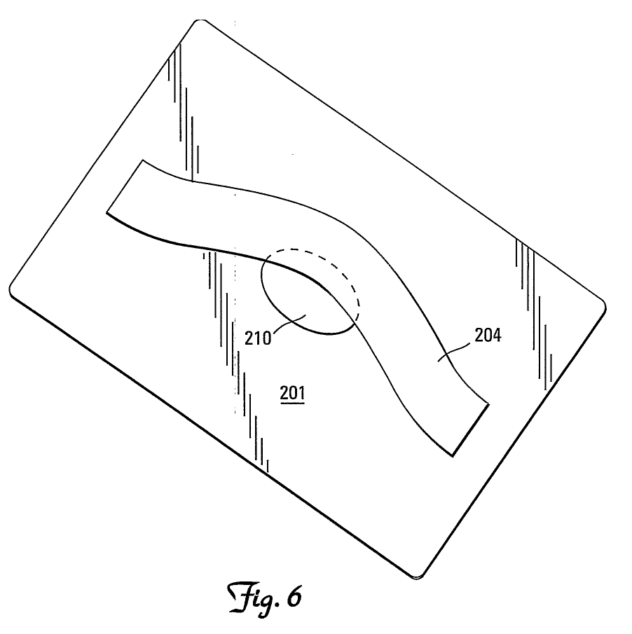

FIGS. 5 and 6 show a pressure bandage 200 according to a second embodiment. The bandage 200 includes a pad 201, backing 202 and a snap-bucklable member 204 generally in accordance with the first embodiment, but with at least two differences. First, the bandage 200 according to the second embodiment can have a small frangible container 210 resembling a gel capsule or an ampoule containing a unit dose, for example a dose of a flowable material such as a liquid medication or other beneficial substance. Second, the snap-bucklable member 204 in this embodiment is an arch-shaped member rather than a dome-shaped member.

The pressure bandage 200 as shown in FIG. 5 has an interior side 206 that generally faces toward the body (not shown) of a wearer (not shown) of the bandage 200. The bandage 200 also has an exterior side 207 that generally faces away from the body (not shown) of the wearer (not shown) of the bandage 200.

By pressing on the arch shaped snap-bucklable member 204 the snap-bucklable member 204 can be inverted, just as the dome shaped snap-bucklable member 104 of the first embodiment was inverted. By inverting the snap-bucklable member 204, pressure is exerted upon on the frangible container 210, causing the container 210 to burst and release liquid medication or other beneficial substance (not shown) stored in the container 210. The liquid could then partially or fully soak into the pad 201. The pad 201 can also be impregnated with a medication or beneficial substance, such as a medication that can act in combination with a liquid released from the container 210.

The frangible container 210 can be made of different materials. Various kinds of gels or plastics can be preferable for many applications. For most applications the container should have a low bursting strength. Glass and brittle plastics are traditionally used for ampoules, but may not be suitable for most applications because of the potentially sharp edges. To make bursting the container 210 easier, a small point (not shown) can be incorporated into the snap-bucklable member 204 to prick the container 210 when pressure is applied to the snap-bucklable member 204.

The bandage 200 as disclosed above can offer advantages including the following. First, the bandage 200 can offer a convenient way to store a liquid such as a medication in a bandage 200 without wetting the entire bandage 200. Second, the bandage 200 can offer a way to release the liquid from the bandage 200 when desired. For example, when the bandage 200 is positioned over a wound (not shown) on the body (not shown). Third, the bandage 200 can offer a way that materials can be mixed for application to the skin (not shown) at an appropriate moment. For example, it may be desirable to have materials mixed after the bandage 200 is applied to the body (not shown). This might be especially true, for example, for materials that do not have a sufficiently long shelf life in a mixed form. Fourth, the bandage 200 can offer a way to use an existing form of container 210 (e.g., a gel capsule) for use with a bandage 200.

### Third Embodiment

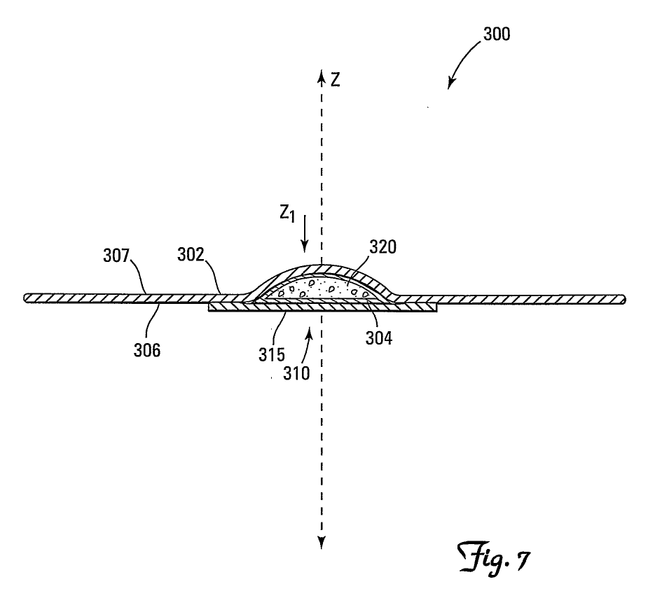

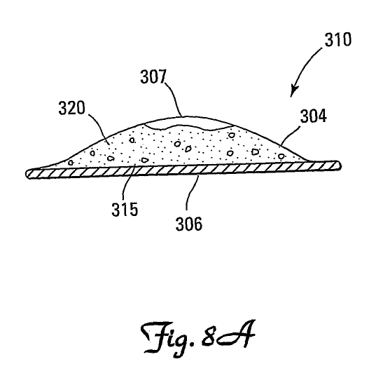

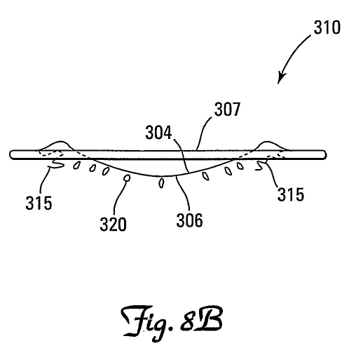

FIGS. 7, 8A and 8B show a bandage 300 according to a third embodiment. The bandage 300 includes a backing 302 and a snap-bucklable member 304 generally in accordance with the first and second embodiments, but with certain differences. First, the bandage 300 according to a third embodiment can be constructed without a pad. Second, the container 310 can be formed differently than the container 210 discussed above in relation to the second embodiment. The container 310 for the flowable material 320 in this embodiment employs a seal 315 over the open end (unnumbered) of the dome-shaped snap-bucklable member 304 to form a retention chamber (unnumbered) for the flowable material 320. The flowable material 320 may be in liquid, powder, or granular form. By applying pressure in the first transverse direction Z₁ to the dome-shaped snap-bucklable member 304, such as pressing down with a finger (not shown), the dome-shaped snap bucklable member 304 can be inverted. FIGS. 8A and 8B show the container 310 formed by the dome-shaped snap bucklable member 304 and the seal 315 in isolation. FIG. 8A shows the sealed container 310 holding a flowable material 320, and FIG. 8B shows that same container 310 after the snap-bucklable member 304 has been inverted so as to break open the seal 315 and release the flowable material 320. The flowable material 320 is released because inverting the snap-buckling member 304 increases pressure on the seal 315 to an extent that the seal 315 burst as shown in FIG. 8B.

The pressure bandage 300 as shown in FIG. 7 has an interior side 306 that generally faces toward the body (not shown) of a wearer (not shown) of the bandage 300. The bandage 300 also has an exterior side 307 that generally faces away from the body (not shown) of the wearer (not shown) of the bandage 300.

The seal 315 can be made of various materials. For many applications a medical grade gelatin film or a wax film can be suitable.

The bandage 300 as disclosed above can offer advantages including the following. First, the bandage 300 can be conveniently manufactured without a pad. The absence of a pad can permit a more direct application of the flowable material 320 to the skin (not shown), can speed the intended action of the flowable material 320, can make manufacture less expensive, and can reduce the bulk of the bandage 300. Second, the dome-shaped snap-bucklable member 304 can form part of the container 310. This can reduce the bulkiness of the bandage 300 and potentially make the release of the flowable material 320 easier.

### Fourth Embodiment

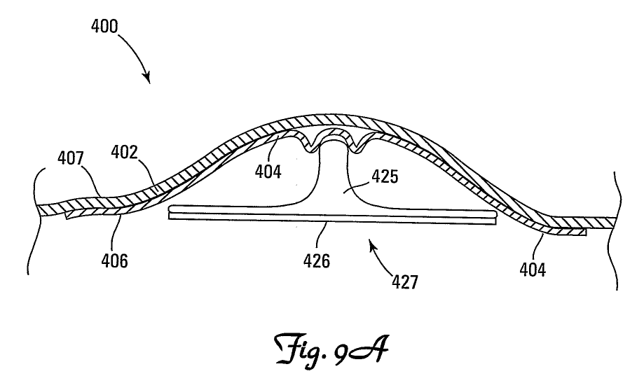

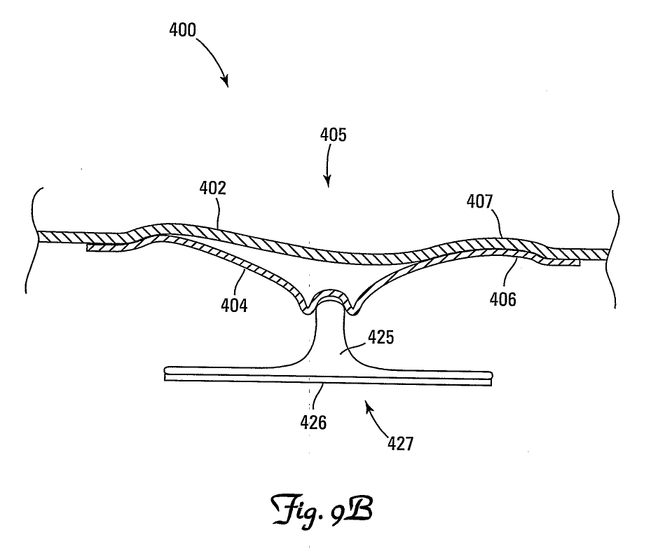

FIGS. 9A and 9B show a portion of a bandage 400 according to a fourth embodiment. The bandage 400 includes a backing 402 and a snap-bucklable member 404 generally in accordance with the first, second and third embodiments, but with certain differences. First, a plunger 425 is provided so as to cooperate with the snap-bucklable member 404. While the snap-bucklable member 404 and the plunger 425 are shown in FIGS. 9A and 9B as two distinct parts, they also could be formed as a unitary piece. Second, the face of the plunger 425 can have a covering 426. The covering 426 can be made of or can contain many different materials. For example, the covering 426 can include a dissolvable solid or semi-solid (e.g., a dissolvable medication or other beneficial substance). The covering 426 can include material such as a medication that can be transmitted transdermally. The covering 426 could be made of material similar to that used for the pad 101 described in relation to the first embodiment.

The pressure bandage 400 as shown in FIGS. 9A and 9B has an interior side 406 that generally faces toward the body (not shown) of a wearer (not shown) of the bandage 400. The bandage 400 also has an exterior side 407 that generally faces away from the body (not shown) of the wearer (not shown) of the bandage 400.

By inverting the snap-bucklable member 404 from concave as shown in FIG. 9A to convex as shown in FIG. 9B, force is exerted upon the plunger 425 in the first transverse direction Z₁.

The fourth embodiment of the bandage 400 can offer advantages including the following. First, the bandage 400 can permit a direct application of medication or other beneficial substances to the skin (not shown) or other target surface (not shown). Second, the bandage 400 can create a relatively substantial amount of pressure at the face 427 of the plunger 425. Third, the pressure can be evenly spread over the face 427 of the plunger 425. Fourth, as mentioned above, the bandage 400 can incorporate solid or semi-solid medications or beneficial substances instead of liquids. This can make packaging easier. Fifth, the combination of pressure and the use of medications or other beneficial substances can speed the administration of the medication or beneficial substances.

### Modifications

The embodiments or examples discussed above can be combined in various ways without departing from the invention. Moreover, the present invention should not be considered limited to the particular examples described above, but rather should be understood to cover all aspects of the invention as fairly set out in the claims arising from this application. For example, while suitable sizes, materials, packaging and the like have been disclosed in the above discussion, it should be appreciated that these are provided by way of example and not of limitation as a number of other sizes, materials, fasteners, and so forth may be used without departing from the invention. Various modifications as well as numerous structures to which the present invention may be applicable will be readily apparent to those of skill in the art to which the present invention is directed upon review of the present specifications. The claims which arise from this application are intended to cover such modifications and structures.

As disclosed the invention concerns a bandage 100, 200, 300 and 400 and in particular a bandage 100, 200, 300 and 400 with a pressure mechanism. Such a bandage 100, 200, 300 and 400 can be used for bandaging parts of the human body (not shown) and applying pressure to a target site (not shown). However, the invention is not limited to such uses. The structure of the bandage 100, 200, 300 and 400 may be useful for other purposes. Other purposes might include the release or application of medications and other useful substances or any other use where a pressure mechanism for a bandage 100, 200, 300 and 400 might be usable and beneficial.

We claim:

1. A pressure bandage for applying pressure to a target site, comprising:
(a) a backing layer having a coating of a pressure-sensitive adhesive on a first major surface of the backing layer, and
(b) a snap-bucklable member coupled to the backing layer and capable of snap-buckling from a concave to a convex configuration after the bandage has been adhesively applied over the target site whereby the convex snap-bucklable member applies continuous pressure to the target site so long as the bandage is worn.

2. A method of applying pressure to a bleeding target site, comprising:
(a) obtaining a pressure bandage, comprising:
(i) a backing layer having a coating of a pressure-sensitive adhesive on a first major surface of the backing layer, and
(ii) a snap-bucklable member coupled to the backing layer and capable of snap-buckling from a concave to a convex configuration,
(b) adhering the pressure bandage over a bleeding target site with the snap-bucklable member positioned over the target site, and
(c) snap-buckling the snap-bucklable member from a concave to a convex configuration.

3. The method of claim 2 wherein the bleeding target site is a skin wound.
